(19) United States
(12) Patent Application Publication
(10) Pub. No.: US 2002/0192686 A1
(43) Pub. Date: Dec. 19, 2002

(54) Method for epigenetic feature selection

(76) Inventors: Peter Adorjan, Berlin (DE); Fabian Model, Berlin (DE)

Correspondence Address: KRIEGSMAN & KRIEGSMAN, 665 Franklin Street, Framingham, MA, 01702, US

(21) Appl. No.: 10/106,269

(22) Filed: Mar. 26, 2002

Related U.S. Application Data
(60) Provisional application No. 60/278,333, filed on Mar. 26, 2001.

(51) Int. Cl. C12Q 1/68; G06F 19/00; G01N 33/48; G01N 33/50

(52) U.S. Cl. 435/6; 702/19; 702/20

(57) ABSTRACT

The present invention provides methods and computer program products for epigenetic feature selection. The invention enables the selection of relevant epigenetic features prior to further data analysis. The invention is preferably used for interpretation of large scale DNA methylation analysis data.

### RELATED APPLICATION

This application claims the priority of U.S. Provisional Application, Serial No. 60/278,333 filed on Mar. 26, 2001. The 60/278,333 application is incorporated herein by reference for all purposes. All cited references are hereby incorporated in their entireties.

### FIELD OF INVENTION

The present invention is related to methods and computer program products for biological data analysis. Specifically, the present invention relates to methods and computer program products for the analysis of large scale DNA methylation analysis.

### BACKGROUND OF THE INVENTION

The levels of observation that have been well studied by the methodological developments of recent years in molecular biology, are the genes themselves, the translation of these genes into RNA, and the resulting proteins. Many biological functions, disease states and related conditions are characterised by differences in the expression levels of various genes. These differences may occur through changes in the copy number of the genomic DNA, through changes in levels of transcription of the genes, or through changes in protein synthesis.

Recently, massive parallel gene expression monitoring methods have been developed to monitor the expression of a large number of genes using mRNA based nucleic acid microarray technology (see, e.g., Lockhart, D. J. et. al., Expression monitoring by hybridization to high density Oligonucleotid arrays, Nature Biotechnology 14:1675-1680, 1996; Lockhart, D. J. et. al., Genomics, gene expression and DNA arrays, Nature 405:827-836, 2000). This technology allows to look at thousands of genes simultaneously, see how they are expressed as proteins and gain insight into cellular processes.

However, large scale analysis using mRNA based microarrays are primarily impeded by the instability of mRNA (Emmert-Buck, T. et al., Am J Pathol. 156, 1109, 2000; U.S. Pat. No. 5,871,928). Also expression changes of only a minimum of a factor 2 can be routinely and reliably detected (Lipshutz, R. J. et. al., High density synthetic oligonucleotide arrays, Nature Genetics 21, 20, 1999; Selinger, D. W. et. al., RNA expression analysis using a 30 base pair resolution Escherichia coli genome array, Nature Biotechnology 18, 1262, 2000). Furthermore, sample preparation is complicated by the fact that expression changes occur within minutes following certain triggers.

An alternative approach is to look at DNA methylation. 5-methylcytosine is the most frequent covalent base modification in the DNA of eukaryotic cells. It plays a role, for example, in the regulation of the transcription, in genetic imprinting, and in tumorigenesis. For example, aberrant DNA methylation within CpG islands is common in human malignancies leading to abrogation or overexpression of a broad spectrum of genes (Jones, P. A., DNA methylation errors and cancer, Cancer Res. 65:2463-2467, 1996). Abnormal methylation has also been shown to occur in CpG rich regulatory elements in intronic and coding parts of genes for certain tumours (Chan, M. F., et al., Relationship between transcription and DNA methylation, Curr. Top. Microbiol. Immunol. 249:75-86,2000). Using restriction landmark genomic scanning, Costello and coworkers were able to show that methylation patterns are tumour-type specific (Costello, J. F. et al., Aberrant CpG-island methylation has non-random and tumor-type-specific patterns, Nature Genetics 24:132-138, 2000). Highly characteristic DNA methylation patterns could also be shown for breast cancer cell lines (Huang, T. H. -M. et al., Hum. Mol. Genet. 8:459-470, 1999).

Therefore, the identification of 5-methylcytosine as a component of genetic information is of considerable interest. However, 5-methylcytosine positions cannot be identified by sequencing since 5-methylcytosine has the same base pairing behaviour as cytosine. Moreover, the epigenetic information carried by 5-methylcytosine is completely lost during PCR amplification.

The state of the art method for large scale methylation analysis (PCT Publication No. WO 99/28498) is based upon the specific reaction of bisulfite with cytosine which, upon subsequent alkaline hydrolysis, is converted to uracil which corresponds to thymidine in its base pairing behaviour. However, 5-methylcytosine remains unmodified under these conditions. Consequently, the original DNA is converted in such a manner that methylcytosine, which originally could not be distinguished from cytosine by its hybridization behaviour, can now be detected as the only remaining cytosine using “normal” molecular biological techniques, for example, by amplification and hybridization to oligonucleotide microarrays or sequencing.

Like mRNA based massive parallel gene expression monitoring experiments, large scale methylation analysis experiments generate unprecedented amounts of information. A single hybridization experiment can produce quantitative results for thousands of CpG positions. Therefore, there is a great need in the art for methods and computer program products to organise, access and analyse the vast amount of information collected using large scale methylation analysis methods.

One approach is to use unsupervised or supervised machine learning methods to analyse large scale methylation data. Unsupervised learning methods as cluster analysis have been applied recently to gene extension analysis (WO 00/28091). However, in large scale methylation analysis the extreme high dimensionality of the data compared to the usually small number of available samples is a severe problem for all classification methods. Therefore, for good performance of the machine learning methods a reduction of the data dimensionality is necessary. This problem is solved by the present invention. The invention provides methods and computer program products for the selection of epigenetic features, as for example the methylation status of CpG positions. Only the corresponding data to these epigenetic features is then subject to machine learning analysis thereby crucially improving the performance of the machine learning analysis.

### SUMMARY OF THE INVENTION

The present invention provides methods and computer program products for selecting epigenetic features. The methods and computer program products are particularly useful in large scale methylation analysis.

In one aspect of the invention methods are provided for selecting epigenetic features comprising the following steps:

In the first step, biological samples containing genomic DNA are collected and stored. The biological samples may comprise cells, cellular components which contain DNA or free DNA. Such sources of DNA may include cell lines, biopsies, blood, sputum, stool, urine, cerebral-spinal fluid, tissue embedded in paraffin such as tissue from eyes, intestine, kidney, brain, heart, prostate, lung, breast or liver, histologic object slides, and all possible combinations thereof.

Next, available phenotypic information about said biological samples is collected and stored, thereby defining a phenotypic data set for the biological samples. The phenotypic information may comprise, for example, kind of tissue, drug resistance, toxicology, organ type, age, life style, disease history, signalling chains, protein synthesis, behaviour, drug abuse, patient history, cellular parameters, treatment history and gene expression.

Next, at least one phenotypic parameter of interest is defined. These defined phenotypic parameters of interest are used to divide the biological samples in at least two disjunct phenotypic classes of interest.

An initial set of epigenetic features of interest is defined. Preferred epigenetic features of interest are, for example, cytosine methylation statuses at selected CpG positions in DNA. This initial set of epigenetic features of interest may be defined using preliminary knowledge data about their correlation with phenotypic parameters.

The defined epigenetic features of interest of the biological samples are measured and/or analysed, thereby generating an epigenetic feature data set.

Next, those epigenetic features of interest and/or combinations of epigenetic features of interest are selected that are relevant for epigenetically based prediction of the phenotypic classes of interest. An epigenetic feature of interest and/or combination of epigenetic features of interest is preferably considered relevant for epigenetically based class prediction if the accuracy and/or the significance of the epigenetically based prediction of said phenotypic classes of interest is likely to decrease by exclusion of the corresponding epigenetic feature data.

Finally, a new set of epigenetic features of interest is defined based on the relevant epigenetic features of interest and/or combinations of epigenetic features of interest generated in the preceding step.

In some embodiments of the invention the steps of measuring and/or analysing the epigenetic features of interest of the biological samples and of selecting the relevant epigenetic features of interest are iteratively repeated based on the epigenetic features of interest defined in the preceding iteration.

In one particularly preferred embodiment, the phenotypic parameters of interest are used to divide the biological samples in two disjunct phenotypic classes of interest. In this embodiment, a machine learning classifier may be used for epigenetically based prediction of the two disjunct phenotypic classes of interest. In another preferred embodiment, the disjunct phenotypic classes of interest are grouped in pairs of classes or pairs of unions of classes and machine learning classifiers may be applied for epigenetically based class prediction to each pair.

In preferred embodiments the selection of the relevant epigenetic features of interest and/or combinations of epigenetic features of interest is done by a) defining a candidate set of epigenetic features of interest and/or combinations of epigenetic features of interest, b) defining a feature selection criterion, c) ranking the candidate set of epigenetic features of interest and/or combinations of epigenetic features of interest according to the defined feature selection criterion and d) selecting the highest ranking epigenetic features of interest and/or combinations of epigenetic features of interest.

The defined candidate set of epigenetic features of interest may be the set of all subsets of the epigenetic features of interest, preferably the set of all subsets of a given cardinality of said defined epigenetic features of interest, in a particularly preferred embodiment the set of all subsets of cardinality 1.

In another preferred embodiment the measured and/or analysed epigenetic feature data set is subject to principal component analysis, the principal components defining a candidate set of linear combinations of the defined epigenetic features of interest.

In other embodiments dimension reduction techniques preferably multidimensional scaling, isometric feature mapping or cluster analysis are used to define the candidate set of epigenetic features of interest and/or combinations of epigenetic features of interest. The cluster analysis may be hierarchical clustering or k-means clustering.

In preferred embodiments which use machine learning classifiers for the prediction of the phenotypic classes of interest based on the epigenetic feature data set the feature selection criterion may be the training error of the machine learning classifier trained on the epigenetic feature data corresponding to the defined candidate set of epigenetic features of interest and/or combinations of epigenetic features of interest. In another preferred embodiment the epigenetic feature selection criterion may be the risk of the machine learning classifier trained on the epigenetic feature data corresponding to the defined candidate set of epigenetic features of interest and/or combinations of epigenetic features of interest. In a further preferred embodiment, the epigenetic feature selection criterion may be the bounds on the risk of the machine learning classifier trained on the epigenetic feature data corresponding to the defined candidate set of epigenetic features of interest and/or combinations of epigenetic features of interest.

In preferred embodiments in which the candidate set of epigenetic features of interest comprises single epigenetic features or single combinations of epigenetic features of interest the epigenetic feature selection criterion may be the use of test statistics for computing the significance of difference of the phenotypic classes of interest given the epigenetic feature data corresponding to the defined candidate set of epigenetic features of interest and/or combinations of epigenetic features of interest. Preferably the statistical test may be a t-test or a rank test, for example a Wilcoxon rank test. In one particularly preferred embodiment, the epigenetic feature selection criterion may be the computation of the Fisher criterion for the phenotypic classes of interest given the epigenetic feature data corresponding to the defined candidate set of epigenetic features of interest and/or combinations of epigenetic features of interest. Furthermore the epigenetic feature selection criterion may be the computation of the weights of a linear discriminant for said phenotypic classes of interest given the epigenetic feature data corresponding to the defined candidate set of epigenetic features of interest and/or combinations of epigenetic features of interest. Particularly preferred linear discriminants are the Fisher discriminant, the discriminant of a support vector machine classifier, the discriminant of a perceptron classifier or the discriminant of a Bayes point machine classifier for said phenotypic classes of interest trained on the epigenetic feature data corresponding to the defined candidate set of epigenetic features of interest and/or combinations of epigenetic features of interest. In yet another embodiment, the epigenetic feature selection criterion may be subjecting the epigenetic feature data corresponding to the defined candidate set of epigenetic features of interest and/or combinations of epigenetic features of interest to principal component analysis and calculating the weights of the first principal component. Moreover, the epigenetic feature selection criterion can be chosen to be the mutual information between the phenotypic classes of interest and the classification achieved by an optimally selected threshold on the given epigenetic feature of interest. Still further, the epigenetic feature selection criterion may be the number of correct classifications achieved by an optimally selected threshold on the given epigenetic feature of interest.

In preferred embodiments in which the epigenetic feature data set is subject to principal component analysis, the principal components defining the candidate set of epigenetic features of interest and/or combinations of epigenetic features of interest, the feature selection criterion can be chosen to be the eigenvalues of the principal components.

In some preferred embodiments, the epigenetic features of interest and/or combinations of epigenetic features of interest selected may be a defined number of the highest ranking epigenetic features of interest and/or combinations of epigenetic features of interest. In other petered embodiments, all except a defined number of lowest ranking epigenetic features of interest and/or combinations of epigenetic features of interest are selected. In yet other preferred embodiments, the epigenetic features of interest and/or combinations of epigenetic features of interest with a feature selection criterion score greater than a defined threshold are selected or all except the epigenetic features of interest and/or combinations of epigenetic features of interest with a feature selection criterion score lesser than a defined threshold are selected.

In preferred embodiments, the iterative method of the invention is repeated until a defined number of epigenetic features of interest and/or combinations of epigenetic features of interest are selected or until all epigenetic features of interest and/or combinations of epigenetic features of interest with a feature selection criterion score greater than a defined threshold are selected.

In particularly preferred embodiments the optimal number of epigenetic features of interest and/or combinations of epigenetic features of interest and/or the optimal feature selection criterion score threshold is determined by crossvalidation of a machine learning classifier on test subsets of the epigenetic feature data.

In some embodiments of the invention, the feature data set corresponding to the defined new set of epigenetic features of interest is used to train a machine teaming classifier.

In another aspect of the invention computer program products are provided. An exemplary computer program product comprises: a) computer code that receives as input an epigenetic feature dataset for a plurality of epigenetic features of interest, the epigenetic feature dataset being grouped in disjunct classes of interest; b) computer code that selects those epigenetic features of interest and/or combinations of epigenetic features of interest that are relevant for machine learning class prediction based on the epigenetic feature data set; c) computer code that defines a new set of epigenetic features of interest based on the relevant epigenetic features of interest and/or combinations of epigenetic features of interest generated in step (b); d) a computer readable medium that stores the computer code. In a preferred embodiment, the computer code repeats step (b) iteratively based on the new defined set of epigenetic features of interest defined in step (c).

Preferably, an epigenetic feature of interest and/or combination of epigenetic features of interest is considered relevant for machine learning class prediction if the accuracy and/or the significance of the class prediction is likely to decrease by exclusion of the corresponding epigenetic feature data.

In one particularly preferred embodiment, the computer code groups the epigenetic feature data set in disjunct pairs of classes and/or pairs of unions of classes of interest before applying the computer code of steps (b) and (c).

In preferred embodiments the computer code selects the relevant epigenetic features of interest and/or combinations of epigenetic features of interest by a) defining candidate sets of epigenetic features of interest and/or combinations of epigenetic features of interest b) ranking the candidate sets of epigenetic features of interest and/or combinations of epigenetic features of interest according to a feature selection criterion and c) selecting the highest ranking epigenetic features of interest and/or combinations of epigenetic features of interest.

The candidate set of epigenetic features of interest the computer code chooses for ranking may be the set of all subsets of the epigenetic features of interest, preferably the set of all subsets of a given cardinality, particularly preferred the set of all subsets of cardinality 1.

In another preferred embodiment the computer code subjects the epigenetic feature data set to principal component analysis, the principal components defining the candidate set of epigenetic features of interest and/or combinations of epigenetic features of interest.

In other embodiments the computer code applies dimension reduction techniques preferably multidimensional scaling, isometric feature mapping or cluster analysis to define the candidate set of epigenetic features of interest and/or combinations of epigenetic features of interest. The cluster analysis may be hierarchical clustering or k-means clustering.

In preferred embodiments the feature selection criterion used by the computer code may be the training error of the machine learning classifier algorithm trained on the epigenetic feature data corresponding to the defined candidate set of epigenetic features of interest and/or combinations of epigenetic features of interest. In another preferred embodiment the epigenetic feature selection criterion is the risk of the machine learning classifier algorithm trained on the epigenetic feature data corresponding to the defined candidate set of epigenetic features of interest and/or combinations of epigenetic features of interest. In a further preferred embodiment, the epigenetic feature selection criterion are the bounds on the risk of the machine learning classifier trained on the epigenetic feature data corresponding to the defined candidate set of epigenetic features of interest and/or combinations of epigenetic features of interest.

In preferred embodiments in which the candidate set of epigenetic features of interest defined by the computer code comprises single epigenetic features or single combinations of epigenetic features of interest the epigenetic feature selection criterion used by the computer code may be the use of test statistics for computing the significance of difference of the classes of interest given the epigenetic feature data corresponding to the chosen candidate set of epigenetic features of interest and/or combinations of epigenetic features of interest. Preferably the statistical test may be a t-test or a rank test, for example a Wilcoxon rank test. In one particularly preferred embodiment, the epigenetic feature selection criterion may be the computation of the Fisher criterion for the classes of interest given the epigenetic feature data corresponding to the defined candidate set of epigenetic features of interest and/or combinations of epigenetic features of interest. Furthermore the epigenetic feature selection criterion may be the computation of the weights of a linear discriminant for the classes of interest given the epigenetic feature data corresponding to the defined candidate set of epigenetic features of interest and/or combinations of epigenetic features of interest. Particularly preferred linear discriminants are the Fisher discriminant, the discriminant of a support vector machine classifier, the discriminant of a perceptron classifier or the discriminant of a Bayes point machine classifier for said phenotypic classes of interest trained on the epigenetic feature data corresponding to the defined candidate set of epigenetic features of interest and/or combinations of epigenetic features of interest. In yet another embodiment, the computer code subjects the epigenetic feature data corresponding to the candidate set of epigenetic features of interest and/or combinations of epigenetic features of interest to principal component analysis and calculates the weights of the first principal component as feature selection criterion. Moreover, the epigenetic feature selection criterion can be chosen to be the mutual information between the classes of interest and the classification achieved by an optimally selected threshold on the given epigenetic feature of interest. Still further, the epigenetic feature selection criterion may be the number of correct classifications achieved by an optimally selected threshold on the given epigenetic feature of interest.

In preferred embodiments in which the the computer code subject the epigenetic feature data set to principal component analysis, the principal components defining the candidate set of epigenetic features of interest and/or combinations of epigenetic features of interest, the feature selection criterion can be chosen to be the eigenvalues of the principal components.

In some preferred embodiments, the epigenetic features of interest and/or combinations of epigenetic features of interest selected by the computer code may be a defined number of the highest ranking epigenetic features of interest and/or combinations of epigenetic features of interest. In other petered embodiments the computer code selects all except a defined number of lowest ranking epigenetic features of interest and/or combinations of epigenetic features of interest. In yet other preferred embodiments, the epigenetic features of interest and/or combinations of epigenetic features of interest with a feature selection criterion score greater than a defined threshold are selected or all except the epigenetic features of interest and/or combinations of epigenetic features of interest with a feature selection criterion score lesser than a defined threshold are selected by the computer code.

In preferred embodiments, the computer code repeats the feature selection steps iteratively until a defined number of epigenetic features of interest and/or combinations of epigenetic features of interest are selected or until all epigenetic features of interest and/or combinations of epigenetic features of interest with a feature selection criterion score greater than a defined threshold are selected.

In particularly preferred embodiments the computer code calculates the optimal number of epigenetic features of interest and/or combinations of epigenetic features of interest and/or the optimal feature selection criterion score threshold by crossvalidation of a machine learning classifier on test subsets of the epigenetic feature data.

In some embodiments of the invention, the computer code uses the feature data set corresponding to the defined new set of epigenetic features of interest to train a machine learning classifier algorithm.

### BRIEF DESCRIPTION OF THE DRAWINGS

FIG. 1 illustrates one embodiment of a process for epigenetic feature selection.

FIG. 2 illustrates one embodiment of an iterative process for epigenetic feature selection.

FIG. 3 shows the results of principal component analysis applied to methylation analysis data. The whole data set (25 samples) was projected onto its first 2 principal components. Circles represent cell lines, triangles primary patient tissue. Filled circles or triangles are AML, empty ones ALL samples.

FIG. 4 Dimension dependence of feature selection performance. The plot shows the generalisation performance of a linear SVM with four different feature selection methods against the number of selected features. The x-axis is scaled logarithmically and gives the number of input features for the SVM, starting with two. The y-axis gives the achieved generalisation performance. Note that the maximum number of principle components corresponds to the number of available samples. Circles show the results for the Fisher Criterion, rectangles for t-test, diamonds for Backward Elimination and Triangles for PCA.

FIG. 5 Fisher Criterion. The methylation profiles of the 20 highest ranking CpG sites according to the Fisher criterion are shown. The highest ranking features are on the bottom of the plot. The labels at the y-axis are identifiers for the CpG dinucleotide analysed. The labels on the x-axis specify the phenotypic classes of the samples. High methylation corresponds to black, uncertainty to grey and low methylation to white.

FIG. 6 Two sample t-test. The methylation profiles of the 20 highest ranking CpG sites according to the two sample t-test are shown. The highest ranking features are on the bottom of the plot. The labels at the y-axis are identifiers for the CpG dinucleotide analysed. The labels on the x-axis specify the phenotypic classes of the samples. High methylation corresponds to black, uncertainty to grey and low methylation to white.

FIG. 7 Backward elimination. The methylation profiles of the 20 highest ranking CpG sites according to the weights of the linear discriminant of a linear SVM are shown. The highest ranking features are on the bottom of the plot. The labels at the y-axis are identifiers for the CpG dinucleotide analysed. The labels on the x-axis specify the phenotypic classes of the samples. High methylation corresponds to black, uncertainty to grey and low methylation to white.

FIG. 8 Support Vector Machine on two best features of the Fisher criterion. The plot shows a SVM trained on the two highest ranking CpG sites according to the Fisher criterion with all ALL and AML samples used as training data. The black points are AML, the grey ones ALL samples. Circled points are the support vectors defining the white borderline between the areas of AML and ALL prediction. The grey value of the background corresponds to the prediction strength.

### DETAILED DESCRIPTION OF THE PREFERRED EMBODIMENTS

The present invention provides methods and computer program products suitable for selecting epigenetic features comprising the steps of:

a) collecting and storing biological samples containing genomic DNA;

b) collecting and storing available phenotypic information about said biological samples; thereby defining a phenotypic data set;

c) defining at least one phenotypic parameter of interest;

d) using said defined phenotypic parameters of interest to divide said biological samples in at least two disjunct phenotypic classes of interest;

e) defining an initial set of epigenetic features of interest;

f) measuring and/or analysing said defined epigenetic features of interest of said biological samples; thereby generating an epigenetic feature data set;

g) selecting those epigenetic features of interest and/or combinations of epigenetic features of interest that are relevant for epigenetically based prediction of said phenotypic classes of interest;

h) defining a new set of epigenetic features of interest based on the relevant epigenetic features of interest and/or combinations of epigenetic features of interest generated in step (g).

In the context of the present invention, “epigenetic features” are, in particular, cytosine methylations and further chemical modifications of DNA and sequences further required for their regulation. Further epigenetic parameters include, for example, the acetylation of histones which, however, cannot be directly analysed using the described method but which, in turn, correlates with DNA methylation. For illustration purpose the invention will be described using exemplary embodiments that analyse cytosine methylation.

Microarray Based DNA Methylation Analysis

In the first step of the method the genomic DNA must be isolated from the collected and stored biological samples. The biological samples may comprise cells, cellular components which contain DNA or free DNA. Such sources of DNA may include cell lines, biopsies, blood, sputum, stool, urine, cerebral-spinal fluid, tissue embedded in paraffin such as tissue from eyes, intestine, kidney, brain, heart, prostate, lung, breast or liver, histologic object slides, and all possible combinations thereof. Extraction may be done by means that are standard to one skilled in the art, these include the use of detergent lysates, sonification and vortexing with glass beads. Such standard methods are found in textbook references (see, e.g., Fritsch and Maniatis eds., Molecular Cloning: A Laboratory Manual, 1989). Once the nucleic acids have been extracted the genomic double stranded DNA is used in the analysis

Next, available phenotypic information about said biological samples is collected and stored. The phenotypic information may comprise, for example, kind of tissue, drug resistance, toxicology, organ type, age, life style, disease history, signalling chains, protein synthesis, behaviour, drug abuse, patient history, cellular parameters, treatment history and gene expression. The phenotypic information for each collected sample will be preferably stored in a database.

At least one phenotypic parameter of interest is defined and used to divide the biological samples in at least two disjunct phenotypic classes of interest. For example the biological samples may be classified as ill and healthy, or tumor cell samples may be classified according to their tumor type or staging of the tumor type.

An initial set of epigenetic features of interest is defined. This initial set of epigenetic features of interest may be defined using preliminary knowledge data about their correlation with phenotypic parameters. In the the illustrated preferred embodiments these epigenetic features of interest will be the cytosine methylation status at CpG dinucleotides located in the promoters, intronic and coding sequences of genes that are known to affect the chosen phenotypic parameters.

In the next step the cytosine methylation status of the selected CpG dinucleotides is measured. The state of the art method for large scale methylation analysis is described in PCT Application WO 99/28498. This method is based upon the specific reaction of bisulfite with cytosine which, upon subsequent alkaline hydrolysis, is converted to uracil which corresponds to thymidine in its base pairing behaviour. However, 5-methylcytosine remains unmodified under these conditions. Consequently, the original DNA is converted in such a manner that methylcytosine, which originally could not be distinguished from cytosine by its hybridization behaviour, can now be detected as the only remaining cytosine using “normal” molecular biological techniques, for example, by amplification and hybridization to oligonucleotide arrays and sequencing. Therefore, in a preferred embodiment, DNA fragments of the pre-treated DNA of regions of interest from promoters, intronic or coding sequence of the selected genes are amplified using fluorescently labelled primers. PCR primers can be designed complementary to DNA segments containing no CpG dinucleotides, thus allowing the unbiased amplification of methylated and unmethylated alleles. Subsequently the amplificates can be hybridised to glass slides carrying for each CpG position of interest a pair of immobilised oligonucleotides. These detection nucleotides are designed to hybridise to the bisulphite converted sequence around one CpG site which is either originally methylated (CG after pre-treatment) or unmethylated (TG after pre-treatment). Hybridisation conditions have to be chosen to allow the detection of the single nucleotide differences between the TG and CG variants. Subsequently ratios for the two fuorescense signals for the TG and CG variants can be measured using, e.g., confocal microscopy. These ratios correspond to the degrees of methylation at each of the CpG sites tested.

Following these steps an epigenetic feature data set X has been generated containing the methylation status of all analysed CpG dinucleotides. This data set may be represented as follows:

X={x¹,x², . . . , xⁱ, . . . ,x^{m}}, with

$${x}^{i} = [ \begin{matrix}{x}_{1}^{i} \\ {x}_{2}^{i} \\ ⋮ \\ {x}_{n}^{i}\end{matrix} ] ,$$

wherein

X is the methylation pattern data set for m samples,

xⁱ is the methylation pattern of sample i,

x₁ⁱ to xₙⁱ are the CG/TG ratios for n analysed CpG positions of sample j.

x₁ to xₙ denote the CG/TG ratios of the n CpG positions, the epigenetic features of interest.

Methylation Based Class Prediction

The next step in large scale methylation analysis is to reveal by means of an evaluation algorithm the correlation of the methylation pattern with phenotypic classes of interest. The analysis strategy generally looks as follows. From many different DNA samples of known phenotypic class of interest (for example, from antibody-labelled cells of the same phenotype, isolated by immunofluorescence), methylation pattern data is generated in a large number of tests, and their reproducibility is tested. Then a machine learning classifier can be trained on the methylation data and the information which class the sample belongs to. The machine learning classifier can then with a sufficient number of training data learn, so to speak, which methylation pattern belongs to which phenotypic class. After the training phase, the machine learning classifier can then be applied to methylation data of samples with unknown phenotypic characteristic to predict the phenotypic class of interest this sample belongs to. For example, by measuring methylation patterns associated with two kinds of tissue, tumor or non-tumor, one obtains labelled data sets that can be used to build diagnostic identifiers.

In a preferred embodiment, where the samples are divided in two phenotypic classes of interest, the task of the machine learning classifier would be to learn, based on the methylation pattern for a given set of training examples X={xⁱ:xⁱεRⁿ} with known class membership Y={yⁱ: yⁱε{a, b}}, where n is the number of CpGs, a and b are the two classes of interest, a discriminant function f:Rⁿ→{a, b}. This discriminant function can then be used to predict the classification of another data set {X′} In machine learning nomenclature the percentage of missclassifications off on the training set {X, Y} is called training error and is usually minimised by the learning machine during the training phase. However, what is of practical interest is the capability to predict the class of previously unseen samples, the so called generalisation performance of the learning machine. This performance is usually estimated by the test error, which is the percentage of misclassifications on an independent test set {X″, Y″} with known classification. The expected value of the test error for all independent test sets is called the risk.

The major problem of training a learning machine with good generalisation performance is to find a discriminant function f which on the one hand is complex enough to capture the essential properties of the data distribution, but which on the other hand avoids over-fitting the data. Numerous machine learning algorithms, e.g., Parzen windows, Fisher's linear discriminant, two decision tree learners, or support vector machines are well known to those of skill in the art. The support vector machine (SVM) (Vapnik, V., Statistical Learning Theory, Wiley, New York, 1998; U.S. Pat. No. 5,640,492; U.S. Pat. No. 5,950,146) is a machine learning algorithm that has shown outstanding performance in several areas of application and has already been successfully used to classify mRNA expression data (see, e.g., Brown, M., et. al., Knowledge-based analysis of microarray gene expression data by using support vector machines, Proc. Natl. Acad. Sci. USA, 97, 262-267, 2000). Therefore, in a preferred embodiment a support vector machine will be trained on the methylation data.

Feature Selection

The major problem of all classification algorithms for methylation analysis is the high dimension of the input space, i.e. the number of CpGs, compared to the small number of analysed samples. The classification algorithms have to cope with very few observations on very many epigenetic features. Therefore, the performance of classification algorithms applied directly to large scale methylation analysis data is generally poor.

The present invention provides methods and computer program products to reduce the high dimension of the methylation data by selecting those epigenetic features or combinations of epigenetic features that are relevant for epigenetically based classification. In this context, an epigenetic feature or a combination of epigenetic features is called relevant, if the accuracy and/or the significance of the epigenetically based classification is likely to decrease by exclusion of the corresponding feature data. For a given classifier, accuracy is the probability of correct classification of a sample with unknown class membership, significance is the probability that a correct classification of a sample was not caused by chance.

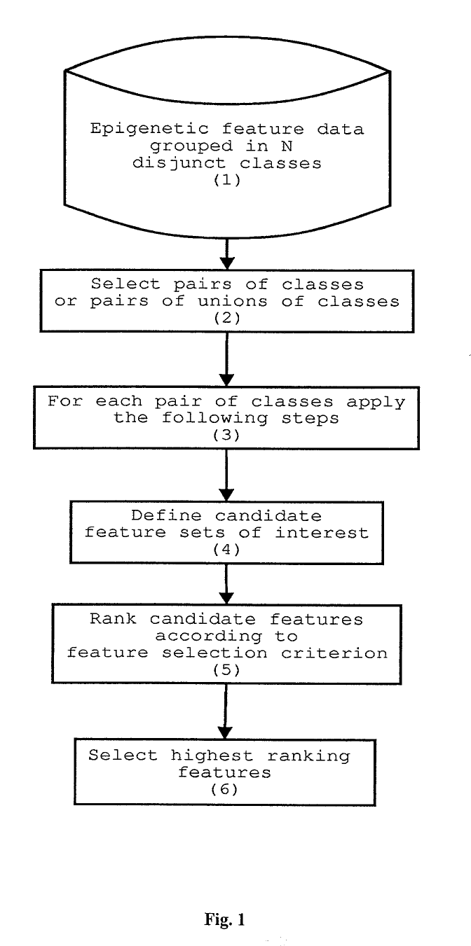

FIG. 1 illustrates a preferred process for the selection of epigenetic features, preferably in a computer system. Epigenetic feature data is inputted in the computer system (1). The epigenetic feature dataset is grouped in at least two disjunct classes of interest, e.g., healthy cell samples and cancer cell samples. If the epigenetic feature data is grouped in more than two disjunct classes of interest pairs of classes or unions of pairs of classes are selected and the feature selection procedure is applied to each of these pairs (2), (3). The reason to look at pairs of classes is that most machine learning classifiers are binary classifiers. Next (4) candidate sets of epigenetic features of interest and/or combinations of epigenetic features of interest are defined. These candidate features are ranked according to a defined feature selection criterion (5) and the highest ranking features are selected (6).

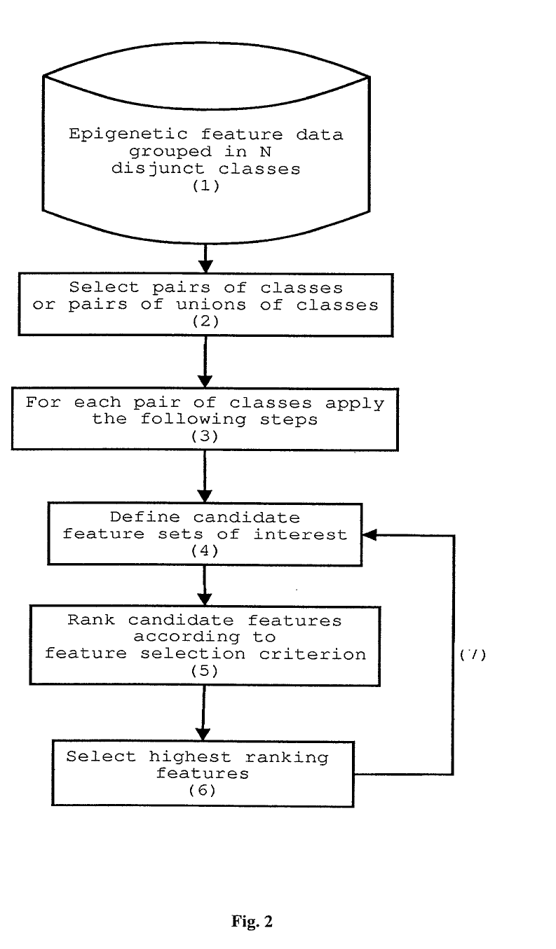

FIG. 2 illustrates an iterative process for the selection of epigenetic features. The process is also preferably performed in a computer system. Epigenetic feature data, grouped in at least two disjunct classes of interest is inputted in the computer system (1). Pairs of disjunct classes or pairs of unions of disjunct classes are selected (2) and (3). Candidate sets of epigenetic features of interest and/or combinations of epigenetic features of interest are defined (4). The candidate features are ranked according to a defined feature selection criterion (5) and the highest ranking features are selected (6). If the number of the selected features is still too big, steps (4), (5) and (6) are repeated starting with the epigenetic feature data corresponding to the selected features of interest selected in step (6). This procedure can be repeated until the desired number of epigenetic features is selected. In every iterative step different candidate feature subsets and different feature selection criteria can be chosen.

In the following the preferred embodiments for defining candidate sets of epigenetic features of interest or combinations of epigenetic features of interest and for defining a feature selection criteria to rank these candidate features will be described in detail.

Candidate Feature Sets

The canonical way to select all relevant features of interest would be to evaluate the generalisation performance of the learning machine on every possible feature subset. This could be done by choosing every possible feature subset for a given set of epigenetic features and estimating the generalisation performance by cross-validation on the training dataset. However, what makes this exhaustive search of the feature space practically useless is the enormous number of

$${\sum }_{k = 0}^{n} ( \begin{matrix}n \\ k\end{matrix} ) = {2}^{n}$$

different feature combinations. Therefore, in a preferred embodiment, the present invention applies a two step procedure for feature selection. First, from the given set of epigenetic features candidate subsets of epigenetic features of interest or combinations of epigenetic features of interest are defined and then ranked according to a chosen feature selection criterion.

In a preferred embodiment, the candidate set of epigenetic features of interest is the set of all subsets of the given epigenetic feature set. In another preferred embodiment, the candidate set of epigenetic features of interest is the set of all subsets of a defined cardinality, i.e. the set of all subsets with a given number of elements. Particularly preferred, the candidate set of epigenetic features of interest is chosen to be the set of all subsets of cardinality 1, i.e. every single feature is selected and ranked according to the defined feature selection criterion.

In other preferred embodiments, dimension reduction techniques are applied to define combinations of epigenetic features of interest. In a particularly preferred embodiment, principal component analysis (PCA) is applied to the epigenetic feature data set. As known to one skilled in the art, for a given data set X, principal component analysis constructs a set of orthogonal vectors (principal components) which correspond to the directions of maximum variance in the data. The single linear combination of the given features that has the highest variance is the first principal component. The highest variance linear combination orthogonal to the first principal component is the second principal component, and so forth (see, e.g., Mardia, K. V., et. al, Multivariate Analysis, Academic Press, London, 1979). To define the candidate set of combinations of epigenetic features of interest the first principal components are chosen.

In another particularly preferred embodiment, multidimensional scaling (MDS) is used to define the candidate features. Contrary to PCA which finds a low dimensional embedding of the data points that best preserves their variance, MDS is a dimension reduction technique that finds an embedding that preserves the interpoint distances (see, e.g., Mardia, K. V., et. al, Multivariate Analysis, Academic Press, London, 1979). To define the candidate set of epigenetic features the epigenetic feature data set X is embedded with MDS in a d-dimensional vector space, the calculated coordinate vectors defining the candidate features. The dimension d of this space is can be fixed and supplied by a user. If not given, one way to estimate the true dimensionality d of the data is to vary d from 1 to n and calculate for every embedding the residual variance of the data. Plotting the residual variance versus the dimension of the embedding the curve generally decreases as the dimensionality d is increased but shows a characteristic “elbow” at which the curve ceases to decrease significantly with added dimensions. This point gives the true dimension of the data (see, e.g., Kruskal, J. B., Wish, M., Multidimensional Scaling, Sage University Paper Series on Quantitative Applications in the Social Sciences, London, 1978, Chapter 3). In another preferred embodiment isometric feature mapping is applied as dimensional reduction technique. Isometric feature mapping is a dimension reduction approach very similar to MDS in searching for a lower dimensional embedding of the data that preserves the interpoint distances. However, contrary to MDS isometric feature mapping can cope with nonlinear structure in the data. The isometric feature mapping algorithm is described in Tenenbaum, J. B., A Global Geometric Framework for Nonlinear Dimensionality reduction, Science 290, 2319-2323, 2000. For the definition of the candidate features, the epigenetic feature data set is embedded in d dimensions using the isometric feature mapping algorithm, the coordinate vectors in the d-dimensional space defining the candidate features. The dimensionality d of the embedding can be fixed and supplied by a user or an optimal dimension can be estimated by looking at the decrease of residual variance of the data for embeddings in increasing dimensions as described for MDS.

In another preferred embodiment, cluster analysis is used to define the candidate set of epigenetic features. Cluster analysis is an effective means to organise and explore relationships in data. Clustering algorithms are methods to divide a set of m observations into g groups so that members of the same group are more alike than members of different groups. If this is successful, the groups are called clusters. Two types of clustering, k-means clustering or partitioning methods and hierarchical clustering, are particularly useful for use with methods of the invention. In signal processing literature partitioning methods are generally denoted as vector quantisation methods. In the following we will use the term k-means clustering synonymously with partitioning methods and vector quantisation methods. k-means clustering partitions the data into a preassigned number of k groups. k is generally fixed and provided by a user. An object (such as a the methylation pattern of a sample) can only belong to one cluster. k-means clustering has the advantage that points are re-evaluated and errors do not propagate. The disadvantages include the need to know the number of clusters in advance, assumption that clusters are round and assumption that the clusters are the same size. Hierarchical clustering algorithms have the advantage to avoid specifying how many clusters are appropriate. They provide the user with many different partitions organised as a tree. By cutting the tree at some level the user may choose an appropriate partitioning. Hierarchical clustering algorithms can be divided in two groups. For a set of m samples, agglomerative algorithms start with m clusters. The algorithm then picks the two clusters with the smallest dissimilarity and merges them. This way the algorithm constructs the tree so to speak from the bottom up. Divisive algorithms start with one cluster and successively split clusters into two parts until this is no longer possible. These algorithms have the advantage that if most interest is on the upper levels of the cluster tree they are much more likely to produce rational clusterings their disadvantage is very low speed. Compared to k-means clustering hierarchical clustering algorithms suffer from early error propagation and no re-evaluation of the cluster members. A detailed description of clustering algorithms can be found in, e.g., Hartigan, J. A., Clustering Algorithms, Wiley, New York, 1975. Having subjected the epigenetic feature data set X to a cluster analysis algorithm, all epigenetic features belonging to the same cluster are combined, e.g., the cluster mean or median is chosen to represent all features belonging to the same cluster, to define the candidate features.

It has to be stressed that in the present invention the described statistical analysis methods aren't used for a final analysis of the large scale methylation data. They are used to define candidate sets of relevant epigenetic features of interest which are then further analysed to select the relevant epigenetic features. These relevant epigenetic features of interest are than used in subsequent analysis.

Feature Selection Criteria

Having defined a candidate set of epigenetic features of interest and/or combinations of epigenetic features of interest, the candidate features are ranked according to preferred selection criteria. In the machine learning literature the feature selection methods are generally distinguished in wrapper methods and filter methods. The essential difference between these approaches is that a wrapper method makes use of the algorithm that will be used to build the final classifier, while a filter method does not. A filter method attempts to rank subsets of the features by making use of sample statistics computed from the empirical distribution.

Some embodiments of the invention make use of wrapper methods. In a preferred embodiment the feature selection criterion may be the training error of a machine learning classifier trained on the epigenetic feature data corresponding to the chosen candidate set of epigenetic features of interest and/or combinations of epigenetic features of interest. For example, if the candidate set of epigenetic features of interest was chosen to be the set of all two-CpG-combinations of the n given CpG positions analysed, i.e.,

{{x₁,x₂},{x₁,x₃}, . . . ,{x₁,xₙ}, . . . ,{x₂,x₃}, . . . ,{xₙ₋₁,xₙ}}

a machine learning classifier is trained for every of the

$$( \begin{matrix}n \\ 2\end{matrix} )$$

two-CpG-combinations on the corresponding methylation pattern data X={xⁱ:xⁱεR²} with known class membership Y={yⁱ:yⁱε{a, b}} and the percentage of misclassifications determined. The two-CpG-subsets are ranked with increasing error.

In another preferred embodiment the feature selection criterion may be the risk of the machine learning classifier trained on the epigenetic feature data corresponding to the defined candidate set of epigenetic features of interest and/or combinations of epigenetic features of interest. The risk is the expected test error of a trained classifier on independent test sets {X′, Y′}. As known to one skilled in the art a common method to determine the test error of a classifier is cross-validation (see, e.g., Bishop, C., Neural networks for pattern recognition, Oxford University Press, New York, 1995). For cross-validation the training set {X, Y} is divided into several parts and in turn using one part as test set, the other parts as training sets. A special form is leave-one-out cross-validation where in turn one sample is dropped from the training set and used as test sample for the classifier trained on the remaining samples. Having evaluated the risk by cross-validation for every element of the defined candidate set of epigenetic features and/or combinations of epigenetic features the elements are ranked by increasing risk.

If for the applied machine learning classsifier theoretical bounds on the risk can be given, these bounds can be chosen as feature selection criteria. A particularly preferred classifier for the analysis of methylation data is the support vector machine algorithm (SVM). For the SVM algorithm bounds on the risk can be derived from statistical learning theory. Details can be found in Vapnik, V. Statistical Learning Theory, Wiley, New York, 1998 or Cristianini, N., Shaw-Taylor, J., An Introduction to Support Vector Machines, Cambridge University Press, Cambridge, 2000. For example, a bound (Theorem 4.24 in Cristianini, Shaw-Taylor) that can be applied as feature selection criterion states that with probability 1-d the risk r of the SVM classifier is bound by

$$r ⩽ \frac{c}{l} ( \frac{{R}^{2} + {z}^{2} log ( 1 / D )}{{D}^{2}} {log}^{2} ( l ) + log ( \frac{1}{l} ) )$$

wherein c is a constant, l is the number of training samples, R is the radius of the minimal sphere enclosing all data points, D is the margin of the support vectors and z is the margin slack vector. R, D, and z are easily derived when training the SVM on every candidate feature subset. Therefore the candidate feature subsets can be ranked with increasing bound values.

Other preferred embodiments of the invention make use of filter methods. If the candidate set of epigenetic features as defined in the preliminary step of the feature selection method of the invention is a set consisting of single epigenetic features combinations of epigenetic features, i.e. {{z₁}{z₂}{z₃} . . . } where the zᵢ are epigenetic features xᵢ or combinations of single epigenetic features xᵢ, test statistics computed from the empirical distribution can be chosen as epigenetic feature selection criteria. A particularly preferred test statistic is a t-test. For example, if the analysed samples can be divided in two classes, say ill and healthy, for every single CpG position xᵢ, the null hypothesis, that the methylation status class means are the same in both classes can be tested with a two sample t-test. The CpG positions can than be ranked by increasing significance value. If there are doubts that the methylation status distribution for any CpG can be approximated by a gaussian normal distribution other embodiments are preferred that use rank test, particularly preferred a Wilcoxon rank test (see, e.g., Mendenhall, W, Sincich, T, Statistics for engineering and the sciences, Prentice-Hall, New Jersey, 1995).

In another preferred embodiment, the Fisher criterion is chosen as feature selection criterion. The Fisher criterion is a classical measure to assess the degree of separation between two classes (see, e.g., Bishop, C., Neural networks for pattern recognition, Oxford University Press, New York, 1995). If, for example, the samples can be divided in two classes, say A and B, the discriminative power of the kth CpG xₖ is given as:

$$J ( k ) = \frac{( {m}_{k}^{A} - {m}_{k}^{B} )}{( {s}_{k}^{2 A} + {s}_{k}^{2 B} )} ,$$

where mₖ^{A/B} is the mean and sₖ^{A/B} is the standard deviation of all sample data values xₖⁱ with y^{j}=A/B. The Fisher criterion gives a high ranking for CpGs where the two classes are far apart compared to the within class variances.

In another preferred embodiment the weights of a linear discriminant used as the classifier are used as the feature selection criterion. The concept of linear discriminant functions is well know to one skilled in the art of neural network and pattern recognition. A detailed introduction can be found, for example, in Bishop, C., Neural networks for pattern recognition, Oxford University Press, New York, 1995. In short, for a two-category classification, if x^{j} is the methylation pattern of sample j, a linear discriminant function z:Rⁿ→R has the form:

z(x^{j})=w^{T}x^{j}+w₀.

The pattern x^{j} is assigned to class C₁ if z(x^{j})>0 and to class C₂ if z(x^{j})≦0. The n-dimensional vector w is called the weight vector and the parameter w₀ the bias. To estimate the weight vector, the discriminant function is trained on a training set. The estimation of the weight vector may, for example, be done calculating a least-squares fit on a training set. Having estimated the coordinate values of the weight vectors, the features can be ranked according to the size of the weight vector coordinates. In a particularly preferred embodiment the weight vector is estimated by Fisher's linear discriminant:

w∝S_{W}⁻¹(m₂−m₁)

where m₁ and m₂ are the mean vectors of the two classes

$${m}_{1} = \frac{1}{{N}_{1}} {\sum }_{i ∈ {C}_{1}} {x}^{i} , {m}_{2} = \frac{1}{{N}_{2}} {\sum }_{i ∈ {C}_{2}} {x}^{i}$$

and

S_{W}=Σ_{iεC}_{₁}(xⁱ−m₁)(xⁱ−m₁)^{T}+Σ_{iεC}_{₂}(xⁱ−m₂)(xⁱ−m₂)^{T}

is the total within-class covaiance matrix.

Another particularly preferred embodiment uses the support vector machine (SVM) algorithm to estimate the weight vector w, see Vapnik, V., Statistical Learning Theory, Wiley, New York, 1998, for a detailed description.

In another preferred embodiment the perceptron algorithm is used to calculate the weight vector w, see Bishop, C., Neural networks for pattern recognition, Oxford University Press, New York, 1995. In a further preferred embodiment the Bayes point algorithm is used to compute the weight vector w as described, e.g., in Herbrich, R., Learning Kernel Classifiers, The MIT Press, Cambridge, Massachusetts, 2002.

In another preferred embodiment PCA is used to rank the defined candidate epigenetic features in the following way: The epigenetic feature data corresponding to the defined candidate set of epigenetic features of interest and/or combinations of epigenetic features of interest is subject to principal component analysis (PCA). Then the ranks of the weights of the first principal component are used to rank the candidate features.

In yet another preferred embodiment, the feature selection criterion is the mutual information between the phenotypical classes of the sample and the classification achieved by an optimally selected threshold on every candidate feature. If {{z₁}{z₂}{z₃} . . . } is the defined set of candidate features where the zᵢ are single epigenetic features xᵢ or combinations of single epigenetic features xᵢ, for every zᵢ a simple classifier is defined by assigning sample j to class C₁ if zᵢ^{j}>bᵢ and to class C₂ if zᵢ^{j}≦bᵢ. The threshold bᵢ is chosen such as to maximise the number of correct classifications on the training data. Note that for every candidate feature the optimal threshold is determined separately. To rank the candidate features the mutual information between each of these classifications and the correct classification is calculated. As known to one skilled in the art the mutual information I of two random variables r and s is given by

I(r,s)=H(r)+H(s)−H(r,s).

H(r)=−Σᵢpᵢ ln pᵢ

is the entropy of random variable r taking the discrete values rᵢ with probability pᵢ and

H(r,s)=−Σᵢⱼpij ln pᵢⱼ

is the joint entropy of the random variables r and s taking the values rᵢ and sⱼ with probability pᵢⱼ (see, e.g., Papoulis, A., Probability, Random Variables and Stochastic Processes, McGraw-Hill, Boston, 1991). In a particularly preferred embodiment, this last step of calculating the mutual information is omitted and the candidate features are ranked according to the number of correct classifications their corresponding optimal threshold classifiers achieve on the training data.

Another preferred embodiment for the choice of the feature selection criterion can be used if the candidate set of epigenetic features of interest and/or combinations of epigenetic features of interest has been defined to be the principal components, subjecting the epigenetic feature data set to PCA as described in the previous section. Then these candidate features can be simply ranked according to the absolute value of the eigenvalues of the principal components.

Selecting the Most Important Features.

Having defined the candidate set of epigenetic features of interest and/or combinations of epigenetic features of interest and ranked theses candidate features according to a preferred feature selection criterion as described in the preceding sections, the final step of the method is to select the most important features from the candidate set.

In a preferred embodiment, a defined number k of highest ranking epigenetic features of interest and/or combinations of epigenetic features of interest is selected from the candidate set. k can be fixed and hard coded in the computer program product or supplied by a user. In another preferred embodiment, all except a defined number k of lowest ranking epigenetic features of interest and/or combinations of epigenetic features of interest are selected from the candidate set. k can be fixed and hard coded in the computer program product or supplied by a user.

In other preferred embodiments, all epigenetic features of interest and/or combinations of epigenetic features of interest with a feature selection criterion score greater than a defined threshold are selected. The threshold can be fixed and hard coded in the computer program. Or, particularly preferred when using the filter methods, the threshold is calculated from a predefined quality requirement like a significance threshold using the empirical distribution of the data. Or, further preferred, the threshold value may be supplied by a user. In other preferred embodiments all epigenetic features of interest and/or combinations of epigenetic features of interest with a feature selection criterion score lesser than a defined threshold are selected, the threshold being fixed and hard coded in the computer program, calculated from the empirical distribution and predefined quality requirements or provided by a user.

In other preferred embodiments, the feature selection steps are iterated until a defined number of epigenetic features of interest and/or combinations of epigenetic features of interest are selected or until all -epigenetic features of interest and/or combinations of epigenetic features of interest with a feature selection score greater than a defined threshold are selected. In every iterative step the same or another feature selection criterion could be chosen. In a similar manner the definition of the new candidate set to rank with the feature selection criterion can be the same in every iterative step or changing with the iterative steps.

A special form of an iterative strategy is known as backward elimination to one skilled in the art. Starting with the full set of epigenetic features as candidate feature set, the preferred feature selection criterion is evaluated and all features selected except the one with the smallest score. These steps are iteratively repeated with the new reduced feature set as candidate set until all except a defined number of features are deleted from the set or all feature with feature selection score lesser than a defined threshold are deleted. Another preferred iterative strategy is known as forward selection to one skilled in the art. Starting with the candidate feature set of all single features, for example, {{x₁}{x₂}{x₃} . . . {xₙ}} the single features are ranked according to the chosen features selection criterion and all are selected for the next iterative step. In the next step the candidate set chosen is the set of subsets of cardinality 2 that include the highest ranking feature from the preceding step. Suppose {x₃} is the highest ranking single feature, the candidate set of features of interest will be chosen as {{x₃,x₁}{x₃,x₂}{x₃,x₄} . . . {x₃,xₙ}}. The feature selection criterion is evaluated and the subset that gives the largest increase in score forms the basis of the candidate set of subsets of cardinality 3 defined in the next iterative step. These steps are repeated until a fixed or user defined cardinality is reached or until there is no further increase in feature selection criterion score from one step to the next.

Another particularly preferred embodiment uses a machine learning classifier to determine the optimal number of epigenetic features of interest and/or combinations of epigenetic features of interest to select. The test error of the classifier is evaluated by cross-validation using in the first stage only the data for the highest ranking feature or feature combination and adding in each successive step one additional feature or feature combination according to the ranking.

Having used the methods of the invention for epigenetic feature selection, the epigenetic feature data corresponding to the selected epigenetic features or combinations of epigenetic features can be used to train a machine learning classifier for the given classification problem. New data to be classified by the trained machine would be preprocessed with the same feature selection method as the training set, before inputting to the classifier. As the example in the following section shows, the methods of the invention greatly improve the performance of machine learning classifiers applied to large scale methylation analysis data.

### EXAMPLE

This example illustrates some embodiments of the method of the invention and its application in DNA methylation based cancer classification. Samples obtained from patients with acute lymphoblastic leukaemia (ALL) or acute myeloid leukaemia (AML) and cell lines derived from different subtypes of leukaemias were chosen to test if classification can be achieved solely based on DNA methylation patterns.

Experimental Protocol

High molecular chromosomal DNA of 6 human B cell precursor leukaemia cell lines, 380, ACC 39; BV-173, ACC 20; MHH-Call-2, ACC 341; MHH-Call-4, ACC 337; NALM-6, ACC 128; and REH, ACC 22 were obtained from the DSMZ (Deutsche Sammlung von Mikroorganismen und Zellkulturen, Braunschweig). DNA prepared from 5 human acute myeloid leukaemia cell lines CTV-1, HL-60, Kasumi-l, K-562 (human chronic myeloid leukaemia in blast crisis) and NB4 (human acute promyelocytic leukaemia) were obtained from University Hospital Charite, Berlin. T cells and B cells from peripheral blood of 8 healthy individuals were isolated by magnetically activated cell separation system (MACS, Miltenyi, Bergisch-Gladbach, Germany) following the manufacturer's recommendations. As determined by FACS analysis, the purified CD4+ T cells were >73% and the CD19+ B cells >90%. Chromosomal DNA of the purified cells was isolated using QIAamp DNA minikit (Qiagen, Hilden, Germany) according to the recommendation of the manufacturer. DNA isolated at time of diagnosis of the peripheral blood or bone marrow samples of 5 ALL-patients (acute lymphoid leukaemia) and 3 AML-patients (acute myeloid leukaemia) was obtained from University Hospital Charite, Berlin.

81 CpG dinucleotide positions located in CpG rich regions of the promoters, intronic and coding sequences of the 11 genes ELK1, CSNK2B, MYCL1, CD63, CDC25A, TUBB2, CD1A, CDK4, MYCN, AR and c-MOS were chosen to be analysed. The 11 genes were randomly selected from a panel of genes representing different pathways associated with tumorigenesis. Total DNA of all samples was treated using a bisulfite solution as described in A. Olek, J. Oswald, J. Walter, Nucleic Acid Res. 24, 5064 (1996). The genomic DNA was digested with MssI (MBI Fermentas, St. Leon-Rot, Germany) prior to the modification by bisulphite. For the PCR amplification of the bisulphite treated sense strand of the 11 genes primers were designed according to the guidelines of Clark and Frommer (S. J. Clark, M. Frommer, in Laboratory Methods for the Detection of Mutations and Polymorphisms in DNA, G. R. Taylor ed., CRC Press, Boca Raton 1997). The PCR primers were designed complementary to DNA segments containing no CpG dinucleotides. This allowed unbiased amplification of both methylated and unmethylated alleles in one reaction. 10 ng DNA was used as template DNA for the PCR reactions. The template DNA, 12.5 pmol or 40 pmol (CY5-labelled) of each primer, 0.5-2 U Taq polymerase (HotStarTaq, Qiagen, Hilden, Germany) and 1 mM dNTPs were incubated with the reaction buffer supplied with the enzyme in a total volume of 20 μl. After activation of the enzyme (15 min, 96° C.) the incubation times and temperatures were 95° C. for 1 min followed by 34 cycles (95° C. for 1 min, annealing temperature (see Supplementary information) for 45 sec, 72° C. for 75 sec) and 72° C. for 10 min.

Oligonucleotides with a C6-amino modification at the 5′ end were spotted with 4-fold redundancy on activated glass slides (T. R. Golub et al., Science 286, 531, 1999). For each analysed CpG position two oligonucleotides N(2-16)-CG-N(2-16) and N(2-16)-TG-N(2-16), reflecting the methylated and non methylated status of the CpG dinucleotides, were spotted and immobilised on the glass array. The oligonucleotide microarrays representing 81 CpG sites were hybridised with a combination of up to 11 Cy5-labelled PCR fragments as described in D. Chen, Z. Yan, D. L. Cole, G. S. Srivatsa, Nucleic Acid Res 27, 389, 1999. Hybridisation conditions were selected to allow the detection of the single nucleotide differences between the TG and CG variants. Subsequently, the fluorescent images of the hybridised slides were obtained using a GenePix 4000 microarray scanner (Axon Instruments). Hybridisation experiments were repeated at least three times for each sample.

Average log CG/TG ratios of the fluorescent signals for the 81 CpG positions were calculated.

Methylation Based Class Prediction

Next support vector machines were trained on this methylation data to learn the classification of samples obtained from patients with acute lymphoblastic leukaemia (ALL) or acute myeloid leukaemia (AML).

In order to evaluate the prediction performance of these SVMs a cross-validation method (Bishop, C., Neural networks for pattern recognition, Oxford University Press, New York, 1995) was used. For each classification task, the 25 samples were partitioned into 8 groups of approximately equal size. Then the SVM predicted the class for the test samples in one group after it had been trained using the 7 other groups. The number of misclassifications was counted over 8 runs of the SVM algorithm for all possible choices of the test group. To obtain a reliable estimate for the test error the number of misclassifications were averaged over 50 different partitionings of the samples into 8 groups.

First, two SVM were trained using all 81 CpG positions as separate dimension. As can be seen in Table I the SVM with linear kernel trained on this 81 dimensional input space had an average test error of 16%. Using a quadratic kernel did not significantly improve the results. An obvious explanation for this relatively poor performance is that we have only 25 data points (even less in the training set) in a 81 dimensional space. Finding a separating hyperplane under these conditions is a heavily under-determined problem. This shows the poor performance of machine learning classifiers applied to large scale methylation analysis data and the great need for the methods provided by the described invention.

Epigenetic Feature Selection

Subsequently some of the preferred embodiments of the invention for selecting epigenetic features were applied and the performance of the SVM for this reduced feature set tested using cross-validation as described above.

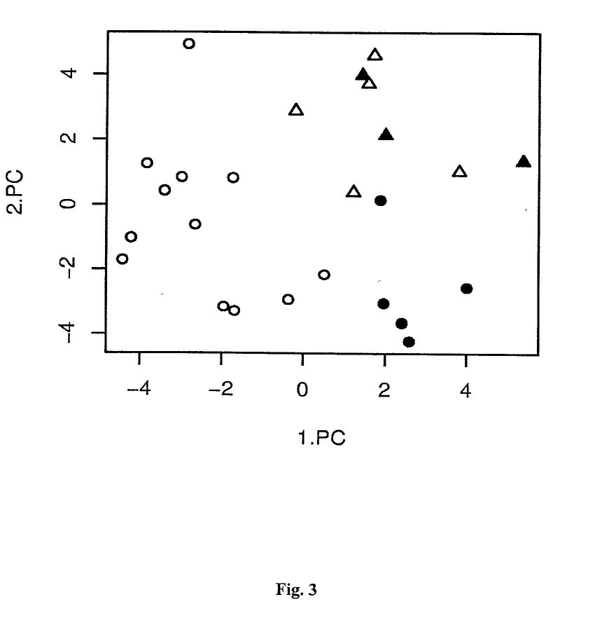

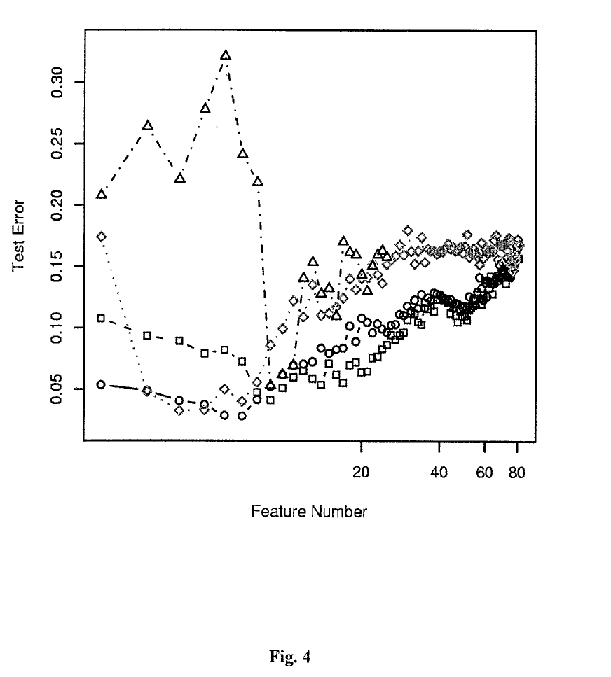

First, PCA was used for epigenetic feature selection. The methylation data for all 81 CpG positions was subject to PCA and the first k principle components selected for k=2 and k=5. Table I shows the results of the performance of SVMs trained and tested on the methylation data projected on this 2- and 5-dimensional feature space. For k=2 the SVM with linear kernel had an average test error of 21% for k=5 an average test error of 28%. The results for a SVM with quadratic kernel were even worse. The reason for this poor performance is that PCA does not necessarily extract features that are important for the discrimination between ALL and AML. It first picks the features with the largest variance, which are in this case discriminating between cell lines and primary patient tissue (see FIG. 3), i.e. subgroups that are not relevant to the classification. As shown in FIG. 4 features carrying information about the leukaemia subclasses appear only from the 9^{th} principal component on.

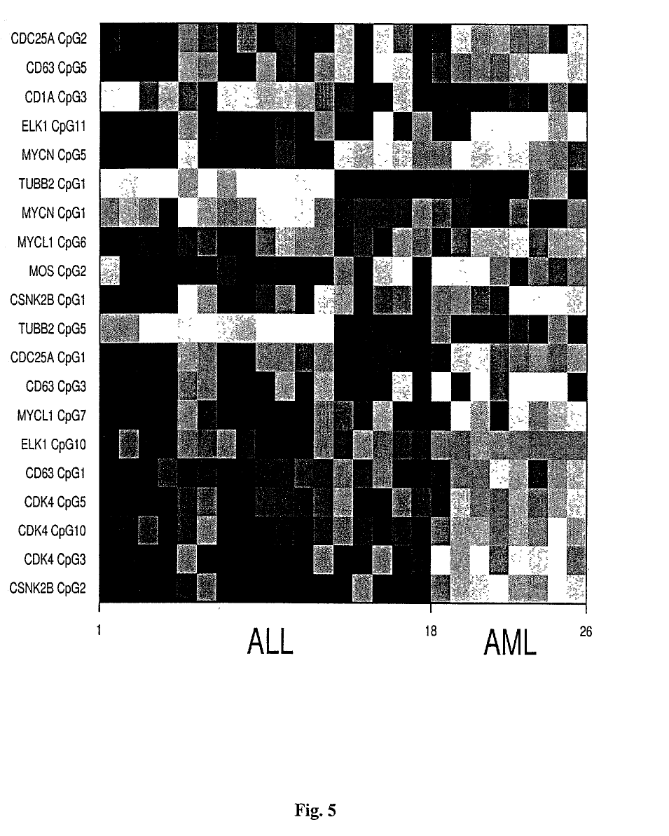

Next all 81 CpG positions were ranked using the Fisher criterion to determine the discriminative power of each CpG for the classification of ALL versus AML. FIG. 5 shows the methylation profiles of the best 20 CpGs . The score increases from bottom to top. SVMs were trained on the 2 and 5 highest ranking CpGs The test error is shown in Table I. The results show a dramatic improvement of generalisation performance compared to no feature selection or PCA. For 5 CpGs the test error decreases from 16% for the linear kernel SVM without feature selection to 3%. FIG. 4 shows the dependence of generalisation performance from the selected dimension k and indicates that especially Fisher criterion (circles) gives dimension independent good generalisation for reasonable small k.

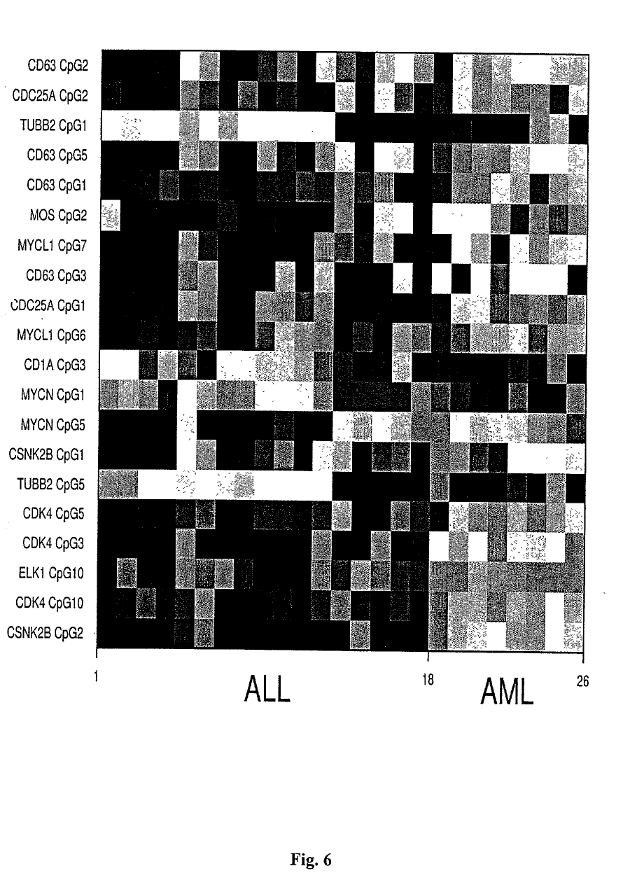

The highest ranking CpG sites according to a two sample t-test are shown in FIG. 6. The ranking of the CpG is very similar to the Fisher criterion. The test errors for SVMs trained on the k highest ranking features for k=2 and k=5 are shown in Table I. Compared to the Fisher criterion the generalisation performance is considerably worse.

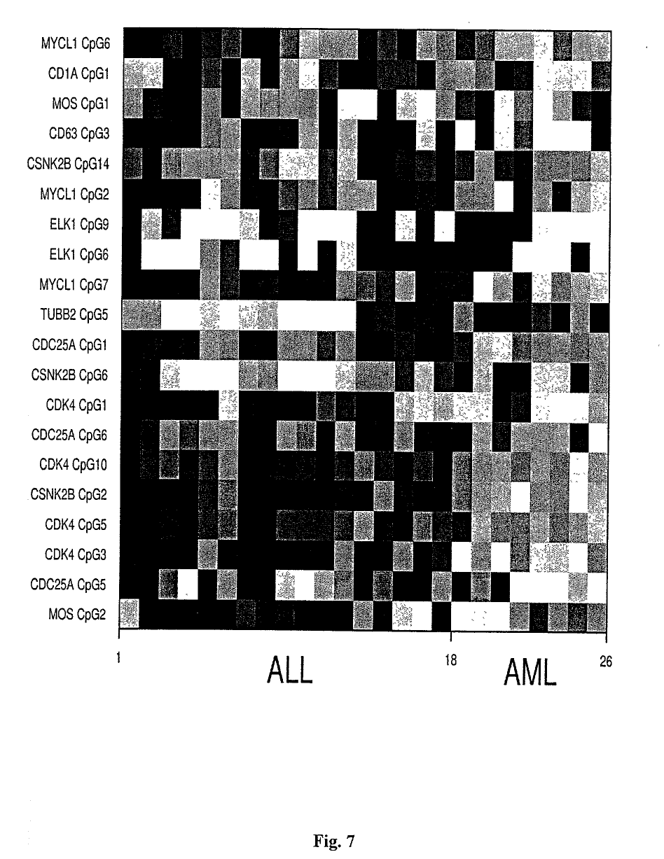

Furthermore the weights of the linear discriminant of the support vector machine algorithm were chosen as feature selection criterion. The candidate features were defined using the backward elimination strategy. The SVM with linear kernel was trained on all 81 CpG and the normal vector w of the separating hyperplane the SVM uses for discrimination calculated. The feature ranking is then simply given by the absolute value of the components of the normal vector. The feature with the smallest component was deleted and the SVM retrained on the reduced feature set. This procedure is repeated until the feature set is empty. The methylation pattern for the highest ranking CpGs according to this selection method is shown in FIG. 7. The ranking differs considerably from the Fisher ant t-test rankings. However, as shown in Table I the generalisation results evaluated when training the SVM on the 2 or 5 highest ranking features werent better than for the Fisher criterion although this method is computationally much more expensive than calculating the Fisher criterion.

Finally the space of all two feature combinations was exhaustively searched to find the optimal two features for classification by evaluating the generalisation performance of the SVM using cross-validation. For every of the

$$( \begin{matrix}81 \\ 2\end{matrix} ) = 3240$$

two CpG combination the leave-one out cross-validation error of a SVM with quadratic kernel was calculated on the training set. From all CpG pairs with minimum leave-one-out error the one with the smallest radius margin ratio was selected. This pair was considered to be the optimal feature combination and was used to evaluate the generalisation performance of the SVM on the test set. The average test error of the exhaustive search method was with 6% the same as the one of the Fisher criterion in the case of two features and a quadratic kernel. For five features the exhaustive computation is already infeasible. In the absolute majority of cross-validation runs the CpGs selected by exhaustive search and Fisher criterion were identical. In some cases suboptimal CpGs were chosen by the exhaustive search method.

It follows that at least for this data set the simple Fisher criterion is the preferable technique for epigenetic feature selection.

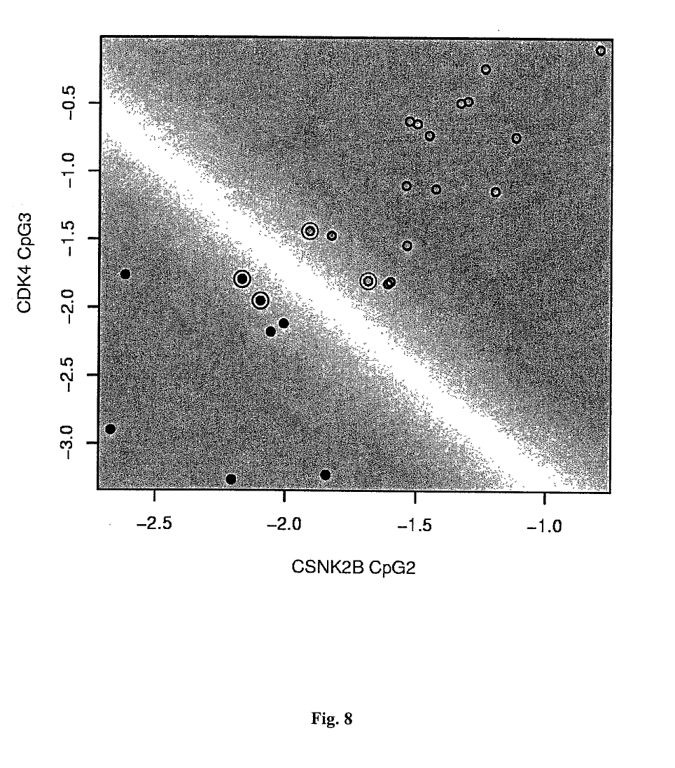

This example clearly shows that microarray based methylation analysis combined with supervised learning techniques and the methods of this invention can reliably predict known tumor classes. FIG. 8 shows the result of the SVM classification trained on the two highest ranking CpG sites according to the Fisher criterion.

TABLE I

| | Training | | Training | |
|---|---|---|---|---|
| | Error | Test Error | Error | Test Error |
| | 2 Features | 2 Features | 5 Features | 5 Features |
| Linear Kernel | | | | |
| Fisher Criterion | 0.01 | 0.05 | 0.00 | 0.03 |
| t-Test | 0.05 | 0.13 | 0.00 | 0.08 |
| Backward Estimation | 0.02 | 0.17 | 0.00 | 0.05 |
| PCA | 0.13 | 0.21 | 0.05 | 0.28 |
| No Feature Selection | 0.00 | 0.16 | — | — |
| Quadratic Kernel | | | | |
| Fisher Criterion | 0.00 | 0.06 | 0.00 | 0.03 |
| t-Test | 0.04 | 0.14 | 0.00 | 0.07 |
| Backward Estimation | 0.00 | 0.12 | 0.00 | 0.05 |
| PCA | 0.10 | 0.30 | 0.00 | 0.31 |
| Exhaustive Search | 0.00 | 0.06 | — | — |
| No Feature Selection | 0.00 | 0.15 | — | — |

What is claimed is:

1. A method for selecting epigenetic features, comprising the steps of:
a) collecting and storing biological samples containing genomic DNA;
b) collecting and storing available phenotypic information about said biological samples;
thereby defining a phenotypic data set,
c) defining at least one phenotypic parameter of interest;
d) using said defined phenotypic parameters of interest to divide said biological samples in at least two disjunct phenotypic classes of interest;
e) defining an initial set of epigenetic features of interest;
f) measuring and/or analysing said defined epigenetic features of interest of said biological samples; thereby generating an epigenetic feature data set;
g) selecting those epigenetic features of interest and/or combinations of epigenetic features of interest that are relevant for epigenetically based prediction of said phenotypic classes of interest;
h) defining a new set of epigenetic features of interest based on the relevant epigenetic features of interest and/or combinations of epigenetic features of interest generated in step (g).

2. The method of claim 1 wherein steps (f) to (g) are repeated based on the new set of epigenetic features of interest defined in step (h).

3. The method of claim 1 or 2 wherein the biological samples comprise cells, cellular components which contain DNA, sources of DNA comprising, for example, cell lines, biopsies, blood, sputum, stool, urine, cerebral-spinal fluid, tissue embedded in paraffin such as tissue from eyes, intestine, kidney, brain, heart, prostate, lung, breast or liver, histologic object slides, and all possible combinations thereof.

4. The method of any one of the claims 1 to 3 wherein the phenotypic information and/or phenotypic parameter of interest are selected from the group comprising kind of tissue, drug resistance, toxicology, organ type, age, life style, disease history, signalling chains, protein synthesis, behaviour, drug abuse, patient history, cellular parameters, treatment history and gene expression and combinations thereof.

5. The method of any one of the claims 1 to 4 wherein the epigenetic features of interest are cytosine methylation sites in DNA.

6. The method of any one of the claims 1 to 5 wherein the initial set of epigenetic features of interest is defined using preliminary knowledge data about their correlation with phenotypic parameters.

7. The method of any one of the claims 1 to 6 wherein an epigenetic feature or a combination of epigenetic features is relevant for epigenetically based prediction of said phenotypic classes of interest if the accuracy and/or the significance of the epigenetically based prediction of said phenotypic classes of interest is likely to decrease by exclusion of the corresponding epigenetic feature data;

8. The method of any one of the claims 1 to 7 wherein said phenotypic parameters of interest are used to divide said biological samples in two disjunct phenotypic classes of interest.

9. The method of claim 8 wherein said epigenetically based prediction of said two disjunct phenotypic classes of interest is done by a machine learning classifier.

10. The method of any one of the claims 1 to 7 wherein from said disjunct phenotypic classes of interest pairs of classes or pairs of unions of classes are selected then subjecting each pair of classes or pair of unions of classes to the method of claims 9.

11. The method of claim 9 wherein said selecting step comprises:
a) defining a candidate set of epigenetic features of interest and/or combinations of epigenetic features of interest,
b) defining a feature selection criterion,
c) ranking the candidate set of epigenetic features of interest and/or combinations of epigenetic features of interest according to said feature selection criterion, and
d) selecting the highest ranking epigenetic features of interest and/or combinations of epigenetic features of interest.

12. The method of claim 11 wherein said candidate set of epigenetic features of interest is the set of all subsets of said defined epigenetic features of interest.

13. The method of claim 11 wherein said candidate set of epigenetic features of interest is the set of all subsets of a given cardinality of said defined epigenetic features of interest.

14. The method of claim 11 wherein said candidate set of epigenetic features of interest is the set of all subsets of cardinality 1 of said defined epigenetic features of interest.

15. The method of claim 11 wherein said epigenetic feature data set is subject to principal component analysis, the principal components defining said candidate set of epigenetic features of interest and/or combinations of epigenetic features of interest.

16. The method of claim 11 wherein said epigenetic feature data set is subject to multidimensional scaling, the calculated coordinate vectors defining said candidate set of epigenetic features of interest and/or combinations of epigenetic features of interest.

17. The method of claim 11 wherein said epigenetic feature data set is subject to isometric feature mapping, the calculated coordinate vectors defining said candidate set of epigenetic features of interest and/or combinations of epigenetic features of interest.

18. The method of claim 11 wherein said epigenetic feature data set is subject to cluster analysis, then combining the epigenetic features of interest belonging to the same cluster to define said candidate set of epigenetic features of interest and/or combinations of epigenetic features of interest.

19. The method of claim 18 wherein said cluster analysis is hierarchical clustering.

20. The method of claim 18 wherein said cluster analysis is k-means clustering.

21. The method of claim 11 wherein said epigenetic feature selection criterion is the training error of the machine learning classifier trained on the epigenetic feature data corresponding to said candidate set of epigenetic features of interest and/or combinations of epigenetic features of interest.

22. The method of claim 11 wherein said epigenetic feature selection criterion is the risk of the machine learning classifier trained on the epigenetic feature data corresponding to said candidate set of epigenetic features of interest and/or combinations of epigenetic features of interest.

23. The method of claim 11 wherein said epigenetic feature selection criterion are the bounds on the risk of the machine learning classifier trained on the epigenetic feature data corresponding to said candidate set of epigenetic features of interest and/or combinations of epigenetic features of interest.

24. The method of any one of the claims 14 to 20 wherein said epigenetic feature selection criterion is the use of test statistics for computing the significance of difference of said phenotypic classes of interest given the epigenetic feature data corresponding to said candidate set of epigenetic features of interest and/or combinations of epigenetic features of interest.

25. The method of claim 24 wherein said statistical test is a t-test.

26. The method of claim 24 wherein said statistical test is a rank test.

27. The method of claim 26 wherein said rank test is a Wilcoxon rank test.

28. The method of any one of the claims 14 to 20 wherein said epigenetic feature selection criterion is the computation of the Fisher criterion for said phenotypic classes of interest given the epigenetic feature data corresponding to said candidate set of epigenetic features of interest and/or combinations of epigenetic features of interest.

29. The method of any one of the claims 14 to 20 wherein said epigenetic feature selection criterion is the computation of the weights of a linear discriminant for said phenotypic classes of interest given the epigenetic feature data corresponding to said candidate set of epigenetic features of interest and/or combinations of epigenetic features of interest.

30. The method of claim 29 wherein said linear discriminant is the Fisher discriminant.

31. The method of claim 29 wherein said linear discriminant is the discriminant of a support vector machine classifier for said phenotypic classes of interest trained on the epigenetic feature data corresponding to said candidate set of epigenetic features of interest and/or combinations of epigenetic features of interest.

32. The method of claim 29 wherein said linear discriminant is the discriminant of a perceptron classifier for said phenotypic classes of interest trained on the epigenetic feature data corresponding to said candidate set of epigenetic features of interest and/or combinations of epigenetic features of interest.

33. The method of claim 29 wherein said linear discriminant is the discriminant of a Bayes Point Machine classifier for said phenotypic classes of interest trained on the epigenetic feature data corresponding to said candidate set of epigenetic features of interest and/or combinations of epigenetic features of interest

34. The method of any one of the claims 14 to 20 wherein said epigenetic feature selection criterion is subjecting the epigenetic feature data corresponding to said candidate set of epigenetic features of interest and/or combinations of epigenetic features of interest to principal component analysis and calculating the weights of the first principal component.

35. The method of any one of the claims 14 to 20 wherein said epigenetic feature selection criterion is the mutual information between said phenotypic classes of interest and the classification achieved by an optimally selected threshold on the given epigenetic feature of interest.

36. The method of any one of the claims 14 to 20 wherein said epigenetic feature selection criterion is the number of correct classifications achieved by an optimally selected threshold on the given epigenetic feature of interest.

37. The method of claim 15 wherein said epigenetic feature selection criterion are the eigenvalues of the principal components.

38. The method of claim 11 wherein a defined number of highest ranking epigenetic features of interest and/or combinations of epigenetic features of interest is selected.

39. The method of claim 11 wherein all except a defined number of lowest ranking epigenetic features of interest and/or combinations of epigenetic features of interest are selected.

40. The method of claim 11 wherein the epigenetic features of interest and/or combinations of epigenetic features of interest with a feature selection criterion score greater than a defined threshold are selected.

41. The method of claim 11 wherein all except the epigenetic features of interest and/or combinations of epigenetic features of interest with a feature selection criterion score lesser than a defined threshold are selected.

42. The method of claim 2 wherein the steps (f) to (g) are repeated until a defined number of epigenetic features of interest and/or combinations of epigenetic features of interest are selected.

43. The method of claim 2 wherein the steps (f) to (g) are repeated until all epigenetic features of interest and/or combinations of epigenetic features of interest with a feature selection criterion score greater than a defined threshold are selected.

44. The method of any one of claims 38 to 43 wherein the optimal number of epigenetic features of interest and/or combinations of epigenetic features of interest and/or the optimal feature selection criterion score threshold is determined by crossvalidation of the classifier on test subsets of the epigenetic feature data.

45. The method of claim 1 or 2 wherein the feature data set corresponding to said defined new set of epigenetic features of interest is used to train a machine learning classifier.

46. A computer program product for selecting epigenetic features comprising
a) computer code that receives as input an epigenetic feature dataset for a plurality of epigenetic features of interest, the epigenetic feature dataset being grouped in disjunct classes of interest;
b) computer code that selects those epigenetic features of interest and/or combinations of epigenetic features of interest that are relevant for machine learning class prediction based on the corresponding epigenetic feature data set;
c) computer code that defines a new set of epigenetic features of interest based on the relevant epigenetic features of interest and/or combinations of epigenetic features of interest generated in step (b);
d) a computer readable medium that stores the computer code.

47. The computer program product of claim 46 comprising computer code that repeats steps (b) based on the new set of epigenetic features defined in step (c).

48. The computer program product of claim 46 or 47 wherein an epigenetic feature of interest and/or combination of epigenetic features of interest is relevant if the accuracy and/or the significance of the machine learning class prediction is likely to decrease by exclusion of the corresponding epigenetic feature data.

49. The computer program product of any one of the claims 46 to 48 wherein said computer code groups the epigenetic feature data set in disjunct pairs of classes and/or pairs of unions of classes of interest before applying the computer code of steps (b) and (c).

50. The computer program product of any one of the claims 46 to 49 wherein said computer code for selecting the relevant epigenetic features of interest and/or combinations of epigenetic features of interest comprises
a) computer code that defines candidate sets of epigenetic features of interest and/or combinations of epigenetic features of interest,
b) computer code that ranks said candidate sets of epigenetic features of interest and/or combinations of epigenetic features of interest according to a feature selection criterion; and
c) computer code that selects the highest ranking epigenetic features of interest and/or combinations of epigenetic features of interest.

51. The computer program product of claim 50 wherein said candidate set of epigenetic features of interest is the set of all subsets of said epigenetic features of interest.

52. The computer program product of claim 50 wherein said candidate set of epigenetic features of interest is the set of all subsets of a given cardinality of said epigenetic features of interest.

53. The computer program product of claim 50 wherein said candidate set of epigenetic features of interest is the set of all subsets of cardinality 1 of said epigenetic features of interest.

54. The computer program product of claim 50 wherein the computer code performs principal component analysis on said epigenetic feature data, the principal components defining said candidate set of epigenetic features of interest and/or combinations of epigenetic features of interest.

55. The computer program product of claim 50 wherein the computer code performs multidimensional scaling on said epigenetic feature data set, the calculated coordinate vectors defining said candidate set of epigenetic features of interest and/or combinations of epigenetic features of interest.

56. The computer program product of claim 50 wherein the computer code performs isometric feature mapping on said epigenetic feature data set, the calculated coordinate vectors defining said candidate set of epigenetic features of interest and/or combinations of epigenetic features of interest.

57. The computer program product of claim 50 wherein the computer code performs cluster analysis on said epigenetic feature data set, then combining the epigenetic features of interest belonging to the same cluster to define said candidate set of epigenetic features of interest and/or combinations of epigenetic features of interest.

58. The computer program product of claim 57 wherein said cluster analysis is hierarchical clustering.

59. The computer program product of claim 57 wherein said cluster analysis is k-means clustering.

60. The computer program product of claim 50 wherein said epigenetic feature selection criterion is the training error of the machine learning classifier trained on the epigenetic feature data corresponding to said candidate set of epigenetic features of interest and/or combinations of epigenetic features of interest.

61. The computer program product of claim 50 wherein said epigenetic feature selection criterion is the risk of the machine learning classifier trained on the epigenetic feature data corresponding to said candidate set of epigenetic features of interest and/or combinations of epigenetic features of interest.

62. The computer program product of claim 50 wherein said epigenetic feature selection criterion are the bounds on the risk of the machine learning classifier trained on the epigenetic feature data corresponding to said candidate set of epigenetic features of interest and/or combinations of epigenetic features of interest.

63. The computer program product of any one of the claims 53 to 59 wherein said epigenetic feature selection criterion is the use of test statistics for computing the significance of difference of said classes of interest given the epigenetic feature data corresponding to said candidate set of epigenetic features of interest and/or combinations of epigenetic features of interest.

64. The computer program product of claim 63 wherein said statistical test is a t-test.

65. The computer program product of claim 63 wherein said statistical test is a rank test.

66. The computer program product of claim 65 wherein said rank test is a Wilcoxon rank test.

67. The computer program product of any one of the claims 53 to 59 wherein said epigenetic feature selection criterion is the computation of the Fisher criterion for said classes of interest given the epigenetic feature data corresponding to said candidate set of epigenetic features of interest and/or combinations of epigenetic features of interest.

68. The computer program product of any one of the claims 53 to 59 wherein said epigenetic feature selection criterion is the computation of the weights of a linear discriminant for said classes of interest given the epigenetic feature data corresponding to said candidate set of epigenetic features of interest and/or combinations of epigenetic features of interest.

69. The computer program product of claim 68 wherein said linear discriminant is the Fisher discriminant.

70. The computer program product of claim 68 wherein said linear discriminant is the discriminant of a support vector machine classifier for said classes of interest trained on the epigenetic feature data corresponding to said candidate set of epigenetic features of interest and/or combinations of epigenetic features of interest.

71. The computer program product of claim 68 wherein said linear discriminant is the discriminant of a perceptron classifier for said classes of interest trained on the epigenetic feature data corresponding to said candidate set of epigenetic features of interest and/or combinations of epigenetic features of interest.

72. The computer program product of claim 68 wherein said linear discriminant is the discriminant of a Bayes Point Machine classifier for said classes of interest trained on the epigenetic feature data corresponding to said candidate set of epigenetic features of interest and/or combinations of epigenetic features of interest.

73. The computer program product of any one of the claims 53 to 59 wherein the computer code performs principal component analysis on said epigenetic feature data corresponding to said candidate set of epigenetic features of interest and/or combinations of epigenetic features of interest said epigenetic feature selection criterion are the weights of the first principal component.

74. The computer program product of any one of the claims 53 to 59 wherein said epigenetic feature selection criterion is the mutual information between said classes of interest and the classification achieved by an optimally selected threshold on the given epigenetic feature of interest.

75. The computer program product of any one of the claims 53 to 59 wherein said epigenetic feature selection criterion is the number of correct classifications achieved by an optimally selected threshold on the given epigenetic feature of interest.

76. The computer program product of claim 54 wherein said epigenetic feature selection criterion are the eigenvalues of the principal components.

77. The computer program product of claim 50 wherein the computer code selects a defined number of highest ranking epigenetic features of interest and/or combinations of epigenetic features of interest.

78. The computer program product of claim 50 wherein the computer code selects all except a defined number of lowest ranking epigenetic features of interest and/or combinations of epigenetic features of interest.

79. The computer program product of claim 50 wherein the computer code selects the epigenetic features of interest and/or combinations of epigenetic features of interest with a feature selection criterion score greater than a defined threshold.

80. The computer program product of claim 50 wherein the computer code selects all except the epigenetic features of interest and/or combinations of epigenetic features of interest with a feature selection criterion score lesser than a defined threshold.

81. The computer program product of claim 47 wherein the steps (b) and (c) are repeated until a defined number of epigenetic features of interest and/or combinations of epigenetic features of interest are selected.

82. The computer program product of claim 47 wherein the computer code repeats the steps (b) and (c) until all epigenetic features of interest and/or combinations of epigenetic features of interest with a feature selection criterion score greater than a defined threshold are selected.

83. The computer program product of any one of claims 77 to 82 wherein the computer code calculates the optimal number of epigenetic features of interest and/or combinations of epigenetic features of interest and/or the optimal feature selection criterion score threshold by crossvalidation of the classifier on test subsets of said epigenetic feature data.

84. The computer program product of claim 46 comprising computer code that uses the epigenetic feature data set corresponding to said defined new set of epigenetic features of interest to train a machine learning classifier.
